(19) United States
(12) Patent Application Publication
(10) Pub. No.: US 2006/0121469 A1
(43) Pub. Date: Jun. 8, 2006

(54) Screening for anti-ovulatory compounds

(76) Inventors: George Malcolm Parker, London (GB); Roger White, London (GB); Goran Leonardsson, Goteborg (SE)

Correspondence Address: ARENT FOX PLLC, 1050 CONNECTICUT AVENUE, N.W., SUITE 400, WASHINGTON, DC, 20036, US

(21) Appl. No.: 10/519,404

(22) PCT Filed: Jul. 4, 2003
(86) PCT No.: PCT/GB03/02876

§ 371 Date: Jan. 31, 2006

(30) Foreign Application Priority Data
Jul. 4, 2002 (GB) 0215496.1

(51) Int. Cl. C12Q 1/68; G01N 33/53

(52) U.S. Cl. 435/6; 435/7.1

(57) ABSTRACT

Screening methods for identifying compounds that are useful as contraceptives are provided. In particular, the compounds inhibit the interaction between steroidogenic factor 1 (SF-1) and nuclear receptor interacting protein (Nrip1) or the up-regulate SF-1 expression.

### TECHNICAL FIELD

This invention relates to screening methods for identifying compounds that are useful as contraceptives. In particular, the invention provides methods of screening for compounds that inhibit the interaction between steroidogenic factor 1 (SF-1) and nuclear receptor interacting protein 1 (Nrip1). The invention also provides methods of screening for compounds that promote expression of SF-1. Preferably, the compounds identified by the methods of the invention promote the transcription of SF-1 regulated genes.

### BACKGROUND ART

Oral contraceptives containing a combination of a progesterone component and an oestrogen component have been used since the 1960's. The progesterone component of the combined pill inhibits ovulation by suppressing luteinising hormone. The oestrogen component acts to inhibit follicle stimulating hormone and luteinising hormone to promote the ovulation inhibitory effect of the progesterone.

These contraceptives work by increasing oestrogen and progesterone levels to suppress the levels of other hormones in the body, so the use of these oral contraceptives is associated with artificial changes in hormone levels. It is possible that these changes may have adverse side-effects. For example, oral contraceptives have been associated with increased risk for myocardial infarction, stroke, venous thromboembolism and possibly breast cancer (Pymar & Crenin, 2001, Semin Reprod Med 19(4): 309-12). There is therefore a need to identify compounds that block ovulation without altering hormone levels. Thus, an object of the invention is the provision of a method for identifying alternative compounds that will inhibit ovulation in mammals.

The nuclear receptor corepressor nuclear receptor interacting protein 1 (Nrip1), also known as RIP140, is known to be essential for female fertility. Female mice null for Nrip1 are viable but infertile because of complete failure to release oocytes at ovulation (White et al, 2000, Nature Medicine, 6:1368-1374). Nrip1 is thought to have a secondary role in maintenance of pregnancy (Leonardsson et al, 2002, Endocrinology, 143(2): 700-707). Prior to the discovery of its role in ovulation, Nrip1 was known to act as a co-activator or co-repressor of a large number of different nuclear hormone receptors. More recently it has been found to modulate transcription of the steroidogenic acute regulatory protein gene (StAR) through interactions with the transcription factors steroidogenic factor 1 (SF-1; also know as Ad4BP) and DAX-1 (Sugawara et al, 2001, Endocrinology 142: 3570-3577). StAR is involved in adrenal gonadal steroid synthesis. However, SF-1 has not been implicated in the control of ovulation.

### DISCLOSURE OF THE INVENTION

It has now been discovered that SF-1 up-regulates the transcription of various genes and that this SF-1 dependent transcription is inhibited in the presence of Nrip1. It is therefore postulated that the absence of ovulation previously seen in Nrip1 null female mice is a result of gene transcription by SF-1 in the absence of any inhibition by Nrip1. Accordingly, it is postulated that promotion of transcription of SF-1 regulated genes, either by inhibiting the interaction between SF-1 and Nrip1 or by promoting expression of SF-1, will inhibit ovulation.

As a first method, the invention therefore provides a method of screening for compounds that inhibit the interaction between SF-1 and Nrip1, said method comprising assessing inhibition of the interaction between SF-1 and Nrip1 in the presence of a candidate compound. Candidate compounds that inhibit the interaction between SF-1 and Nrip1 may have contraceptive efficacy.

Preferably, the method of screening for compounds that inhibit the interaction between SF-1 and Nrip1 comprises the steps of:
(a) mixing Nrip1, SF-1 and one or more candidate compounds;
(b) incubating the mixture to allow Nrip1, SF-1 and the candidate compound(s) to interact; and
(c) assessing whether the interaction between Nrip1 and SF-1 is inhibited.

The mixing of Nrip1, SF-1 and candidate compound in step (a) may be done in any order.

As a second method, the invention provides a method of screening for compounds that promote the expression of SF-1, said method comprising assessing the level of expression of SF-1 in the presence of a candidate compound. Candidate compounds that promote the expression of SF-1 may override the inhibition of SF-1 by Nrip1 and hence have contraceptive efficacy.

Direct Screening for Inhibitors of SF-1:Nrip1 Interaction:

Inhibition of the SF-1/Nrip1 interaction in the presence of candidate compounds may be assessed directly. Various methods for direct detection of protein/protein interactions are available.

For example, one or both of SF-1 and Nrip1 may be labelled with a fluorescent label such that the interaction between SF-1 and Nrip1 may be detected by an intrinsic fluorescence change which occurs when an SF-1:Nrip1 complex is formed or disrupted. For example, SF-1 may be joined to a fluorescence resonance energy transfer (FRET) donor and Nrip1 to a FRET acceptor (or vice versa) such that, when SF-1 and Nrip1 interact, stimulation of the FRET donor excites the FRET acceptor causing it to emit photons. Interaction may be also be detected by fluorescent labelling of SF-1 and/or Nrip1 such that fluorescence is quenched when they form a complex.

Other methods for assessing interaction between SF-1 and Nrip1 may include using NMR to determine whether an SF-1:Nrip1 complex is present when a candidate compound is present.

The presence of a SF-1:Nrip1 complex may also be detected as a band at a particular position when run on a gel. Disruption of the complex by addition of a candidate compound may be detected by the presence of bands at different positions on the gel.

Interaction of Nrip1 and SF-1 may also be assessed by detecting the accessibility of peptide sequences (e.g. epitopes) on Nrip1 and/or SF-1 that are masked when the two proteins form a complex. For example, motifs on Nrip1 that interact with nuclear receptors have been identified in WO98/49561, Lee et al (Mol Cell Biol, 1998, 18(11): 6745-44) and in Wei et al (J. Biol. Chem., 2001, 276(19): 16107-12). A lack of interaction between Nrip1 and SF-1 may therefore be determined by detection of such motifs, for example, using antibodies.

Another method of assessing whether interaction between Nrip1 and SF-1 is inhibited by a candidate compound may involve immobilising Nrip1 on a solid surface and assaying for the presence of free SF-1 in the presence of SF-1 and a candidate compound. If interaction between SF-1 and Nrip1 is inhibited by the candidate compound, free SF-1 will be detected. The SF-1 may be labelled to facilitate detection. This type of assay may also be carried with SF-1 being immobilised on the solid surface. Interaction between the immobilised and the free protein may also be monitored by a process such as surface plasmon resonance.

Other methods are described by Sugawara et al, 2001, Endocrinology 142: 3570-3577.

Indirect Screening for Inhibitors of SF-1:Nrip1 Interaction Using Two-Hybrid Systems:

Whether the interaction between SF-1 and Nrip1 is inhibited in the presence of a candidate compound may also be assessed indirectly. One indirect method of screening for inhibition of the interaction between SF-1 and Nrip1 in the presence of a candidate compound involves using a two-hybrid system. SF-1 may be fused to an activation domain of a transcription factor and Nrip1 to a DNA-binding domain of a transcription factor (or vice versa), such that interaction between SF-1 and Nrip1 promotes the transcription of a reporter gene in a cell.

The invention provides a method of screening for compounds that inhibit the interaction between SF-1 and Nrip1, said method comprising:
a) contacting a cell containing a nucleic acid molecule comprising a promoter operatively linked to a reporter gene with: (i) a first fusion protein comprising one of SF-1 and Nrip1 fused to the activation domain of a transcription factor, (ii) a second fusion protein comprising the other of SF-1 and Nrip1 fused to the DNA-binding domain of a transcription factor, and (iii) a candidate compound; and
b) assessing the level of expression of the reporter gene,
   wherein interaction between SF-1 and Nrip1 promotes transcription of the reporter gene by activating said promoter.

This method may be used to assess interaction between Nrip1 and SF-1 in any eukaryotic cell. Preferably, the method is used to assess the interaction between Nrip1 and SF-1 in a yeast cell or a mammalian cell. Where the candidate compound is an organic compound and a yeast two-hybrid system is being used, the permeability of the yeast cell wall is preferably enhanced e.g. by using chemicals such as polymyxin B.

The level of expression of a reporter gene in the two-hybrid system is indicative of the level of interaction between SF-1 and Nrip1. A candidate compound that inhibits the interaction between SF-1 and Nrip1 decreases or abolishes the level of expression of the reporter gene.

Preferably, the reporter gene is easily assayed. For example, the reporter gene may give a detectable signal, such as a visible signal. The reporter gene may encode a protein which gives a visible signal itself, or which catalyses a reaction which gives a visible change e.g. a fluorescent protein or an enzyme. The reporter gene may encode an enzyme such as a beta-galactosidase or a peroxidase, both of which are commonly used with coloured substrates and/or products. The reporter gene may encode a green fluorescent protein (GFP) or a fluorescent derivative thereof such as YFP or CFP (see Prasher et al, 1995, Trends Genet 11(8): 320). The reporter gene may encode a luminescent protein, such as luciferase.

The reporter gene may drive DNA replication (Vasavada et al, 1991, PNAS, 88:10686-10690) in the cell or may encode a drug resistance marker (Fearon et al, 1992, PNAS 89: 7958-7962).

The reporter gene may encode a protein that enables positive selection of cells in which the interaction between SF-1 and Nrip1 is inhibited. For example, the reporter gene may encode a protein that is toxic or cytostatic so that only cells that do not express the protein are able to survive or grow. As a result, the only cells to survive are those in which the candidate compound inhibits the interaction between SF-1 and Nrip1 so that the reporter gene is not expressed. Examples of reporter genes of this type that may be used in yeast include URA3, LYS2 and CYH2 (see Vidal et al, 1996, PNAS, 93: 10315-10320). The protein encoded by the reporter gene may also prevent cell growth in the absence or presence of a particular amino acid or other component in cell media. For example, the reporter gene may encode a DNA-binding protein, Tn10 tetracycline, which represses transcription of a TetRop-HIS3 gene so that yeast cells in which the reporter gene is expressed do not grow in the absence of histidine (see Shih et al, 1996, PNAS, 93: 13896-13901). In contrast, yeast cells in which the interaction between Nrip1 and SF-1 has been disrupted do not express TN10 tetracycline and are consequently able to grow in the absence of histidine.

The proteins encoded by the reporter genes may be in the form of fusion proteins. Methods for the generation of fusion proteins are standard in the art and will be known to the skilled reader. For example, most general molecular biology, microbiology recombinant DNA technology and immunological techniques can be found in Sambrook et al., (Molecular Cloning, A Laboratory Manual, Cold Harbor-Laboratory Press, Cold Spring Harbor, N.Y., 2000) or Ausubel et al., (Current Protocols in Molecular Biology, Wiley Interscience, NY, 1991).

Other Indirect Screening Methods for Inhibitors of SF-1:Nrip1 Interaction:

Because SF-1 is a transcription factor which is inhibited by Nrip1, interaction between SF-1 and Nrip1 may also be assessed indirectly by means of a reporter gene under the control of a promoter which is regulated by SF-1. The invention provides a method of screening for compounds that inhibit the interaction between SF-1 and Nrip1, said method comprising:
a) contacting a nucleic acid molecule, comprising an SF-1-regulated promoter operatively linked to a reporter gene, with one or more candidate compounds, in the presence of SF-1 and Nrip1; and
b) assessing the level of expression of the reporter gene.

This method employs a nucleic acid molecule comprising a promoter operatively linked to a reporter gene, such that transcription of the reporter gene is under the control of the promoter. These nucleic acid molecules are referred to as reporter constructs. Such reporter constructs are already known from the prior art e.g. Koskimies et al. (2002) Endocrinology 143:909-919 describes reporter constructs which express luciferase under the control of a RLF promoter, with transcription regulated by SF-1.

The promoter in the construct is an SF-1 regulated promoter from which SF-1 is able to promote transcription of the reporter gene. SF-1 regulated promoters contain at least one gonadotroph specific element (GSE) to which SF-1 binds to initiate transcription (Bryan et al, 1999, J. Molec. Endocrin., 22:241-249). Preferably, the GSE comprises the sequence RRAGGTCA or YCAAGGYYR.

The SF-1 regulated promoter may be derived from the region upstream of any gene the transcription of which is regulated by SF-1. Genes that are linked to an SF-1 regulated promoter in nature from which the SF-1 regulated promoter may be derived include aromatase, lutenising hormone beta, follicle stimulating hormone, StAR, cholesterol side-chain cleavage enzyme, DAX-1, anti-mullerian hormone, kallikrein and RLF (see Kosikimies et al, 2002, Endocrinology 143(3): 909-919). Mutant SF-1 promoters may also be used e.g. those described by Koskimies et al. [supra].

The reporter gene controlled by the promoter may be a gene which is regulated by SF-1 in nature (see above). In such situations, the reporter construct preferably includes the reporter gene and its natural upstream regulatory sequences.

Preferably, however, the promoter controls transcription of a heterologous reporter gene which is easily assayed, as described above for two-hybrid methods. However, in the two-hybrid system, compounds that inhibit the interaction between SF-1 and Nrip1 are detected as a result of a decrease in the expression of the reporter gene; in contrast, where the transcriptional activity of SF-1 is itself detected, compounds that inhibit the interaction result in an increase in the expression of the reporter gene because repression of SF-1 activity by Nrip1 is inhibited.

For example, where the reporter gene is a fluorescent protein, inhibition of the interaction between Nrip1 and SF-1 may be detected by an increase in fluorescent protein expression (cf two-hybrid system). Where the method is carried out in a cell and the reporter gene is a toxic protein, only cells in which there is no inhibition of the interaction between Nrip1 and SF-1 survive i.e. killing is the “positive” result.

The proteins encoded by the reporter genes may be in the form of fusion proteins as described above. For example, genes that give a visible signal may be fused downstream of a gene that is linked to the promoter in the construct in nature.

Indirect Screening Methods for Up-Regulation of SF-1 Expression:

Screening for compounds that promote the expression of SF-1 may also be conducted indirectly using a reporter gene. In general, such methods operate as described above, with modifications as necessary. Thus the invention provides a method of screening for compounds that promote the expression of SF-1, said method comprising:
a) contacting a nucleic acid molecule comprising a promoter from a SF-1 gene operatively linked to a reporter gene with one or more candidate compounds; and
b) assessing the level of expression of the reporter gene.

Compounds that promote SF-1 expression result in increased expression of the reporter gene.

The promoter is from an SF-1 gene and may be derived from the region upstream of any SF-1 gene. Preferably, the promoter is derived from the region upstream of a mammalian SF-1 gene. The promoter may be derived from the region upstream of the SF-1 gene endogenous to a cell or tissue in which an assay is performed. The promoter may be derived from the region upstream of the human SF-1 gene.

The reporter gene give an easily-assayed product, as described above. Alternatively, the reporter may be SF-1 itself, with the level of SF-1 expression detected indirectly using a nucleic acid molecule comprising a SF-1 regulated promoter operatively linked to a second reporter gene, as described above.

Vectors Comprising Reporter Constructs:

Reporter constructs used indirect screening methods of the invention may be in the form of a viral vector or a non-viral vector. Preferably, the nucleic acid molecules used in these methods of the invention are in the form of a conventional non-viral vector, such as a plasmid. Where these indirect screening methods are conducted in cell-based or tissue-based assays, the introduction of the non-viral vector into the animal cells may be carried out by any method known in the art including dextran-mediated transfection, calcium phosphate precipitation, polybrene-mediated transfection, protoplast fusion, electroporation, encapsulation of polynucleotides in liposomes or direct microinjection of the DNA into nuclei etc.

Use of Nucleic Acid Molecules:

The invention provides the use of a reporter construct, as described above, in a method of screening for anti-ovulatory compounds.

Systems for Carrying Out Screening Methods

The methods of the invention may be carried out in cell-free systems or in cells or tissues.

In particular, the indirect screening methods described above may be carried out in a cell-free system, in a cell or in a tissue. The cell-free system must contain all the necessary components for transcription of the reporter gene where the level of expression is detected by measuring mRNA levels, and all the necessary components for transcription and translation of the reporter gene where the level of expression is assessed by measuring protein levels. Where the method of screening for compounds that promote expression of SF-1 is carried out in a cell-free transcription system, for instance, the system must comprise the transcription factors necessary for transcription of the SF-1 gene.

It is preferred that the methods of screening of the invention be conducted in cell-free systems since this facilitates high-throughput screening of candidate compounds.

Indirect screening methods of the invention are preferably carried out in eukaryotic cells, such as mammalian (e.g. human) or yeast cells. They may also be performed in mammalian (e.g human) tissues. A typical cell is an ovary cell or a Leydig cell. A typical tissue is ovarian tissue.

When the indirect method of screening for compounds that inhibit the interaction of Nrip1 and SF-1 using a nucleic acid molecule comprising an SF-1 regulated promoter is carried out in a cell, the cell should preferably express both Nrip1 and SF-1 endogenously. If Nrip1 and SF-1 are not endogenously expressed, they may be introduced into the cell using a viral or non-viral vector encoding the Nrip1 or SF-1. Preferably, the Nrip1 and SF-1 are introduced into the cell in the form of plasmids.

Assessing Level of Expression of Reporter Gene:

The level of expression of the reporter gene may be assessed by measuring the level of a mRNA transcribed from the reporter gene or the level of protein translated after its transcription.

Measuring Level of mRNA

The level of mRNA transcribed from a reporter gene can be assessed, for example, by traditional blotting techniques described in Sambrook et al [supra]. Messenger RNA can be purified and separated using gel electrophoresis. The nucleic acids on the gel are then blotted onto a solid support, such as nitrocellulose. The solid support is exposed to a labelled probe and then washed to remove any unhybridized probe. Next, the duplexes containing the labelled probe are detected. Typically, the probe is labelled with a radioactive moiety.

Alternatively, the level of mRNA transcribed from the reporter gene may be detected by PCR-based methods. The mRNA transcribed from the reporter gene may be specifically amplified using primers that only bind to the mRNA with the amplified mRNA being detected using the blotting methods described above. The level of transcription of the reporter gene could be also detected using fluorescence resonance energy transfer (FRET) through fluorophores coupled to two oligonucleotides that are complementary to the mRNA transcribed from the reporter gene. (see Wouters et al, 2001, Trends in Cell Biology 11,203-211).

As the cell-free system, cell or tissue will contain DNA from which the mRNA is transcribed, it is preferred to use a RNA-specific detection technique or to focus on sequence present in the mRNA transcription but not in the DNA (e.g. splice junctions, polyA tail etc.). The methods of the invention may comprise an initial step of: extracting mRNA from the cell-free system, cell or tissue; removing DNA from the cell-free system, cell or tissue; and/or disrupting DNA but not mRNA in the cell-free system cell or tissue.

Methods for selectively extracting RNA from biological samples are well known and include methods based on guanidinium buffers, lithium chloride, acid phenol:chloroform extraction, SDS/potassium acetate etc. After total RNA has been extracted, mRNA may be enriched for example using oligo-dT techniques. Methods for removing DNA from biological samples include DNase digestion. Methods for removing DNA encoding the reporter gene transcript but not the RNA transcribed from it will use an agent which is specific to a sequence within the DNA.

Measuring Level of Protein

Measurement of mRNA levels is not ideal in high throughput screening methods. The invention also provides that reporter gene expression can be assessed by measuring protein levels.

The level of protein expressed from the reporter gene can be conveniently measured by using an antibody which binds to the protein encoded by the reporter gene. Following removal of unbound antibody, the level of the protein encoded by the reporter gene can be determined by assessing the level of the antibody bound to it. This may be done by labelling the antibody that binds to the protein or by using a second labelled antibody which binds to the first antibody.

Where the reporter gene encodes a protein that provides a visible signal, the level of expression of the reporter gene is preferably assessed by detecting the visible signal. For example, where the reporter gene encodes a fluorescent protein such as GFP, or an enzyme such as luciferase, the level of expression may be assessed by fluorescence/luminescence detection. Where the reporter gene encodes a protein that is toxic or cytostatic, the level of expression may be assessed by looking at cell survival or cell growth.

Reference Standards:

A reference standard (e.g a control), is typically needed in order to detect whether the interaction between SF-1 and Nrip1 is inhibited in the first method of the invention and whether the expression of SF-1 is up-regulated in the second method. In order to detect whether a candidate compound inhibits the interaction between SF-1 and Nrip1, the interaction between SF-1 and Nrip1 in the presence of a candidate compound may be compared with the interaction between SF-1 and Nrip1 in the absence of a candidate compound. In order to detect whether the expression of SF-1 is promoted by a candidate compound, the level of expression of SF-1 in the presence of a candidate compound may be compared with the level of expression of SF-1 in the absence of the candidate compound.

The reference may have been determined before performing the method of the invention, or may be determined during (e.g. in parallel) or after the method has been performed. It may be an absolute standard derived from previous work.

SF-1 and Nrip1:

The methods of the invention may use SF-1 and Nrip1 derived from any eukaryote. Preferably, they use SF-1 and Nrip1 derived from an animal, such as a mammal.

The SF-1 gene has been cloned and sequenced in a number of mammalian species including mouse (Tsukiyama T et al, 1992, Mol Cell Biol 12:1286-1291; Ikeda Y et al, 1993, Mol Endocrin, 7: 852-860) and sheep (NCBI database entry AAG24622.1 GI:10945629). The human gene for the transcription factor SF-1 has also been cloned and sequenced (Wong et al, 1996, J. Mol Endocrinol, 17(2): 139-47) and its protein structure has been studied (Oba et al, 1996, Biochem Biophys Res Commun, 226(1): 261-7). The SF-1 gene is evolutionarily conserved in vertebrates and invertebrates and homologues of the vertebrate genes are found in a number of invertebrates including Drosophila and hookworm (see Parker & Schimmer, 1997, Endocrine Reviews 18(3): 361-376 for review).

The Nrip1 gene has been cloned in a number of mammalian species including humans (Cavailles et al, 1995, EMBO J., 14:3741-3751) and mouse (Lee et al, 1998, Mol Cell Biol, 18: 6745-55).

Preferably, Nrip1 and SF-1 used in a method of the invention are both derived from the same mammal. The Nrip1 and SF-1 proteins are preferably both human proteins and, when SF-1-regulated reporter constructs are used, the promoter is preferably a human promoter.

Reference in the methods of the invention to the use of SF-1 and Nrip1 includes the use of variants of SF-1 which retain the ability to interact with Nrip1, and vice versa. Suitable variants are known from the literature e.g. a number of human SF-1 protein variants are known to be generated by alternative splicing (Kramer et al, 1998, Gene, 211(1): 29-37) and these variants may be used in the methods of the invention. Other variants include natural biological variants, allelic variants, fragments, and mutants containing amino acid substitutions, insertions or deletions from the wild type sequence.

Fragments of natural SF-1 that interact with Nrip1 and fragments of Nrip1 that interact with SF-1 may also be used in the methods of the invention. Motifs in Nrip1 that interact with nuclear receptors are disclosed in WO98/49561, Lee et al (Mol Cell Biol, 1998, 18(11): 6745-44) and in Wei et al (J. Biol. Chem., 2001, 276(19): 16107-12). References to the use of SF-1 and Nrip1 in the methods of the invention also includes the use of such fragments of SF-1 and Nrip1.

Polypeptides that are structurally similar to SF-1 and Nrip1, or to fragments of Nrip1 and SF-1 that retain the ability to interact, may also be used in the methods of the invention. These may be derived from natural SF-1 or Nrip1 or they may be prepared synthetically or using techniques of genetic engineering. In particular, synthetic molecules that are designed to mimic the tertiary structure of SF-1 or Nrip1 and in particular the domains of SF-1 and Nrip1 that interact may be used in the methods of the invention. References to the use of SF-1 and Nrip1 in the methods of the invention include the use of polypeptides that are structurally similar to SF-1 and Nrip1, or to fragments thereof.

References to the use SF-1 and Nrip1 in the methods of the invention also include the use of fusion proteins comprising SF-1 or Nrip1, fusion proteins comprising variants or fragments thereof, or fusion proteins comprising polypeptides that are structurally similar to SF-1 or Nrip1 or to fragments of SF-1 or Nrip1. Such fusion proteins are particularly useful in two-hybrid methods.

Candidate Compounds

Candidate Compounds Used in Screening Methods:

Typical candidate compounds for use in all the screening methods of the invention include, but are not restricted to, peptides, peptoids, proteins, lipids, metals, small organic molecules, RNA aptamers, antibiotics and other known pharmaceuticals, polyamines, antibodies or antibody derivatives (e.g. antigen-binding fragments, single chain antibodies including scFvs, etc), and combinations or derivatives thereof. Small organic molecules have a molecular weight of about more than 50 and less than about 2,500 daltons, and most preferably between about 300 and about 800 daltons. Candidate compounds may be derived from large libraries of synthetic or natural compounds. For instance, synthetic compound libraries are commercially available from MayBridge Chemical Co. (Revillet, Cornwall, UK) or Aldrich (Milwaukee, Wis.). Alternatively, libraries of natural compounds in the form of bacterial, fungal, plant and animal extracts may be used. Additionally, candidate compounds may be synthetically produced using combinatorial chemistry either as individual compounds or as mixtures.

Preliminary Screening for Candidate Compounds:

In some instances, it may be desirable to conduct a preliminary screening step to reduce the number of candidate compounds used in the methods of the invention. Compounds that bind to Nrip1 or SF-1 individually are inherently more likely to inhibit interaction between SF-1 and Nrip1 than those that do not.

The invention therefore provides a method of screening for compounds that inhibit the interaction between Nrip1 and SF-1 further comprising the preliminary step of screening for candidate compounds by identifying compounds that bind to either Nrip1 or SF-1.

Candidate compounds may be pre-screened in this way using the two-hybrid systems. In particular, candidate compounds may be compounds identified as binding to the signature motif in Nrip1 identified in WO98/49561, Lee et al (Mol Cell Biol, 1998, 18(11): 6745-44) or in Wei et al (J. Biol. Chem., 2001, 276(19): 16107-12). Pre-screening may also be used with antibodies (or derivatives).

It has been suggested that the binding of Nrip1 to nuclear receptors may be inhibited by acetylation of the domains in Nrip1 that interacts with the receptor (Vo et al, 2001, Mol Cell Biol, 21(18): 6181-8). Candidate compounds may therefore be compounds identified as acetylating Nrip1 in a pre-screen.

Candidate compounds may also be compounds identified in a pre-screen as binding to the carboxy-terminal transcriptional activation domain of SF-1 (Ito et al, 1998, Mol. Endocrinol., 12(2):290-301).

In vivo Confirmation of Function of Compounds Identified

Once a compound has been identified as an inhibitor of the interaction between SF-1 and Nrip1 or as a promoter of the expression of SF-1, it may be desirable to perform further experiments to confirm the in vivo anti-ovulatory function of the compound.

The invention therefore provides a method of assessing the anti-ovulatory effect of a compound obtained or obtainable by any of the methods described above in vivo comprising administering the compound to a female mammal and assessing the effect on ovulation. The female mammal may be any species of mammal but is preferably a monkey, pig, rabbit or mouse. Tests in non-humans may be used.

The methods of screening for compounds that inhibit the interaction between SF-1 and Nrip or that promote the expression of SF-1 may take place in a different geographical location from the method for assessing the anti-ovulatory effect of the compounds identified.

Compounds Identified by Screening Methods

The invention further provides a compound that inhibits the interaction between Nrip1 and SF-1 obtained or obtainable by any of the methods described above. The invention also provides a compound that promotes expression of SF-1 obtained or obtainable by any of the methods described above. Preferably, the compounds of the invention are organic compounds.

There is also provided a composition comprising a compound that inhibits the interaction between Nrip1 and SF-1 or a compound that promotes expression of SF-1, obtained or obtainable by any of the methods described above.

Compounds that are found to inhibit ovulation may be useful contraceptives in their own right or may be lead compounds for the development of new contraceptives. They may also be useful in contraceptive research.

Pharmaceutical Uses of Compounds Identified

Once a compound has been identified using one of the methods of the invention, it may be necessary to conduct further work on its pharmaceutical properties. For example, it may be necessary to alter the compound to improve its pharmacokinetic properties or bioavailability. The invention extends to any compounds identified by the methods of the invention which have been altered to improve their pharmacokinetic properties, and to composition comprising those compounds.

The invention further provides compounds obtained or obtainable using the methods of the invention, and compositions comprising those compounds, for use as contraceptives. The invention also provides the use of a compounds obtained or obtainable using the methods of the invention, or compositions comprising those compounds in the manufacture of a medicament to prevent ovulation. A contraceptive method comprising administering a compound obtained or obtainable by any one of the methods of the invention, or a composition comprising such a compound, to a mammal, preferably a human, is also provided.

### BRIEF DESCRIPTION OF THE DRAWINGS

FIG. 1 shows kallikrein (KLK) expression analysis in murine wild-type and Nrip knock-out ovarian samples. FIG. 1A shows a Northern blot. FIG. 1B shows real-time PCR analysis of tissue KLK, with clear up-regulation in homozygous knockout mice. FIG. 1C shows in situ staining of wild-type tissue and FIG. 1D shows the same staining for a homozygous knockout.

FIG. 2 shows TaqMan real-time PCR analysis of RLF/INSL3 expression in ovarian samples.

FIG. 3 shows SF-1 regulated expression of TKLK and RLF/INSL3 in transiently transfected 293 cells.

### EXAMPLES

Various aspects and embodiments of the present invention will now be described in some detail. It will be appreciated that modification of detail may be made without departing from the spirit and scope of the invention.

Kallikrein Expression Analysis in Wild-Type and Nrip Knock-Out Ovarian Samples.

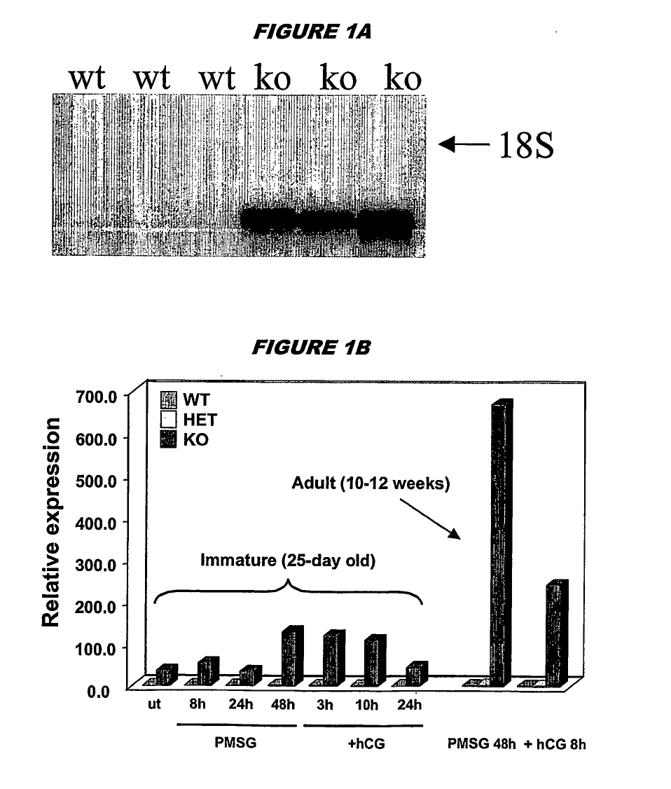

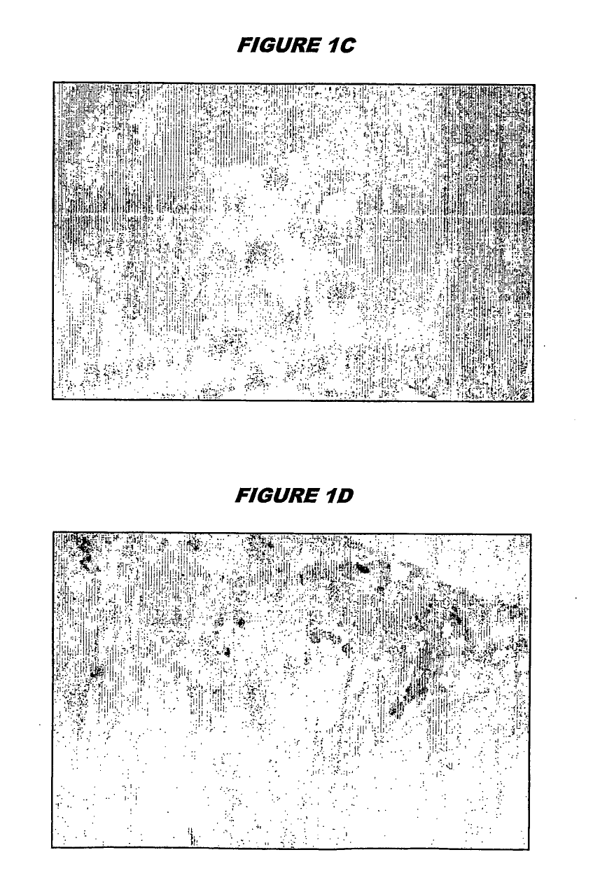

Northern blot, TaqMan real-time PCR and In Situ hybridisation analysis were used to detect the expression pattern of the kallikrein gene in Nrip1 knock-out mice. As shown in FIG. 1, Northern Blot analysis (1A) could demonstrate kallikrein (KLK) expression only in Nrip1 knock-out RNA samples. TaqMan real-time analysis (1B) showed that the mRNA levels of KLK during the ovulatory process is dramatically elevated in immature and to an even higher degree in mature knock-out mice compared to wild-type and heterozygous mice. In Situ hybridisation analysis (1C & 1D) showed that kallikreins are expressed in ovarian theca/interstitial cells.

TaqMan Real-Time PCR Analysis of RLF/INSL3 Expression in Ovarian Samples.

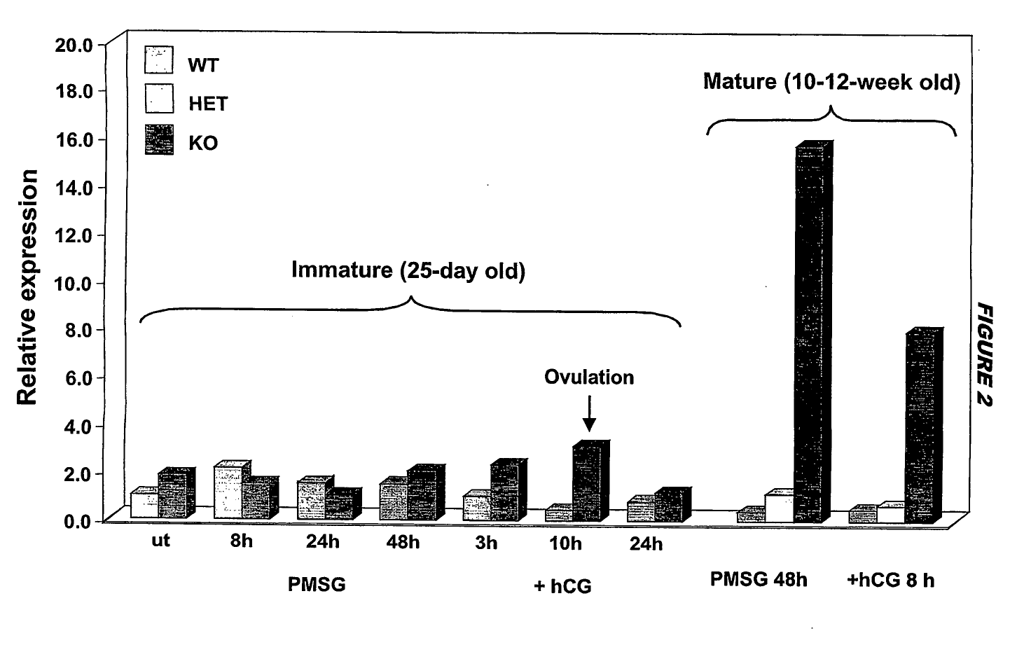

As shown in FIG. 2, TaqMan real-time PCR analysis demonstrated that, as for kallikreins, the mRNA level of RLF/INSL3 is dramatically elevated in KO mice (red) compared to wild-type and heterozygous mice.

Transient Transfection Analysis of Kallikrein and RLF/INSL3 Promoter Activity.

The mouse RLF gene contains several binding sites for SF-1, which have been found to be functionally important in transient transfections of leydig (mLTC) and luteinized ovary (KK1) cells (Koskimies et al, 2002, Endocrinol., 143(3): 909-919). When the mouse kallikrein promoter was analysed, it was also found to contain several potential SF-1 binding sites.

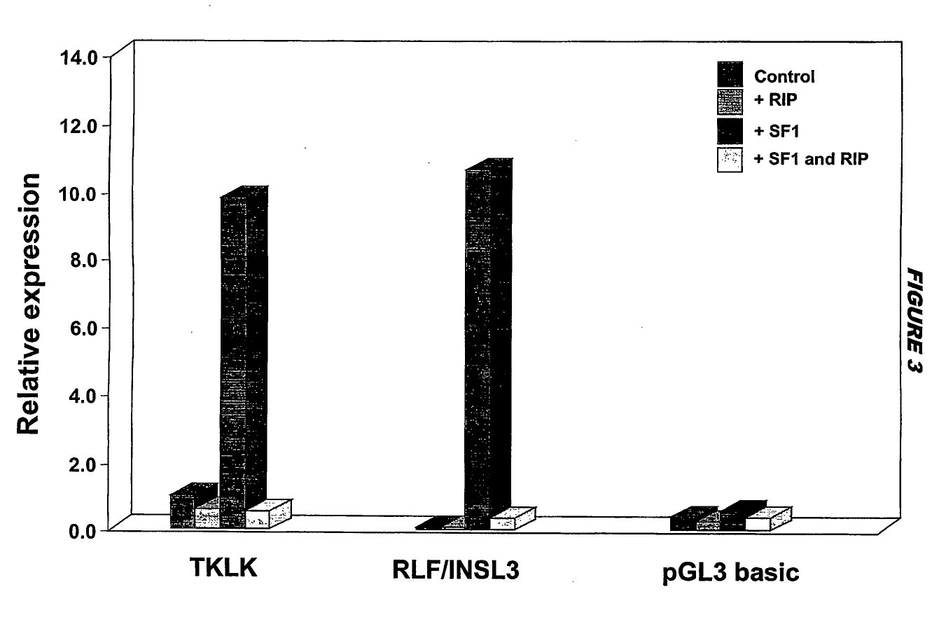

To determine whether SF-1 could regulate expression of RLF and KLK from their respective promoters and to what extent any expression was modulated by Nrip1, transient transfections of 293 cells with luciferase reporter constructs containing DNA fragments of the respective promoters were performed. A transient transfection of 293 cells with an empty reporter vector (pGL3-basic) was also performed. Expression plasmids containing SF-1 and Nrip1 were co-transfected as indicated in FIG. 3.

As shown in FIG. 3, expression from both the KLK and RLF/INSL3 constructs can be activated with co-transfection of SF-1. However, addition of Nrip1 essentially represses all SF-1 mediated up-regulation, suggesting that Nrip1 has the potential regulate SF-1 mediated expression. Lack of Nrip1 activity in Nrip1 KO mice may therefore result in over-expression of SF-1 regulated genes in vivo.

The reporter constructs comprising an SF-1 regulated promoter linked to a luciferase reporter gene can be used to screen for candidate compounds that reverse the inhibition of SF-1 by Nrip1. FIG. 3 shows the level of expression of the reporter gene in the presence of Nrip1 and SF-1. The experiments described above can be repeated in the presence of a candidate compound to identify compounds that increase the level of expression of the reporter gene. Candidate compounds that increase the level of expression of the reporter gene may have anti-ovulatory properties.

Further experimental details can be found in Parker et al. (2003) FEBS Lett 546:149-153.

1. A method of screening for compounds that inhibit the interaction between SF-1 and Nrip1, said method comprising assessing inhibition of the interaction between SF-1 and Nrip1 in the presence of one or more candidate compounds.

2. A method according to claim 1 comprising:
a) mixing Nrip1, SF-1 and one or more candidate compounds;
b) incubating the mixture to allow Nrip1, SF-1 and the candidate compound(s) to interact; and
c) assessing whether interaction between Nrip1 and SF-1 is inhibited.

3. A method according to claim 1 comprising:
a) contacting a cell containing a nucleic acid molecule comprising a promoter operatively linked to a reporter gene with: (i) a first fusion protein comprising one of SF-1 and Nrip1 fused to the activation domain of a transcription factor, (ii) a second fusion protein comprising the other of SF-1 and Nrip1 fused to the DNA-binding domain of a transcription factor, and (iii) a candidate compound; and
b) assessing the level of expression of the reporter gene,
wherein interaction between SF-1 and Nrip1 promotes transcription of the reporter gene by activating said promoter.

4. A method according to claim 1 comprising:
a) contacting a nucleic acid molecule, comprising an SF-1 regulated promoter operatively linked to a reporter gene, with one or more candidate compound(s) in the presence of SF-1 and Nrip1; and
b) assessing the level of expression of the reporter gene.

5. A method of screening for compounds that up-regulate expression of SF-1, said method comprising:
a) contacting a nucleic acid molecule comprising a promoter from a SF-1 gene operatively linked to a reporter gene with a candidate compound; and
b) assessing the level of expression of the reporter gene.

6. A method according to any one of claims 3 to 5, wherein the promoter controls transcription of a reporter gene with which it is linked in nature.

7. A method according to any one of claims 3 to 6, wherein expression of the reporter gene gives a detectable signal.

8. A method according to claim 7, wherein the reporter gene encodes a fluorescent protein or an enzyme.

9. A reporter gene according to claim 7, wherein the reporter gene encodes a toxic or cystostatic protein.

10. A method according to any preceding claim, wherein said method is carried out in a cell free system, a cell or a tissue.

11. A method according to any one of claims 3 to 10, wherein the nucleic acid molecule is in the form of a non-viral vector.

12. A method according to any one of claims 3 to 11, wherein the step of assessing the level of expression of the reporter gene comprises measuring the level of mRNA transcribed from the reporter gene.

13. A method according to any one of claims 3 to 11, wherein the step of assessing the level of expression of the reporter gene comprises measuring the level of protein translated after transcription of the reporter gene.

14. A method according to any preceding claim, comprising the further steps of administering to a female mammal a candidate compound found to inhibit the interaction between SF-1 and Nrip1, or to up-regulate SF-1 expression, and assessing its effect on ovulation.

15. A compound that inhibits the interaction between Nrip1 and SF-1, obtained or obtainable by a method of any one claims 1 to 4 or 6 to 13.

16. A compound that up-regulated expression of SF-1, obtained or obtainable by a method of any one of claims 5 to 13.

17. A method of assessing the anti-ovulatory effect of a compound according to claim 15 or claim 16, comprising administering the compound to a female mammal and assessing its effect on ovulation.

18. A composition comprising a compound according to claim 15 or claim 16.

19. A compound according to claim 15 or claim 16 for use as a contraceptive.

20. Use of a compound according to claim 15 or claim 16 in the manufacture of a medicament to prevent ovulation.

21. A contraceptive method comprising administering a compound according to any claim 15 or claim 16, or a composition according to claim 18, to female mammal.
